(19) United States
(12) Patent Application Publication
(10) Pub. No.: US 2010/0063587 A1
(43) Pub. Date: Mar. 11, 2010

(54) Metal lower eyelid pocket reducer

(76) Inventor: Kun Yuan Tong, Suwanee, GA (US)

Correspondence Address: Kun Yuan Tong, 2842 Lowe Court, Suwanee, GA, 30024, US

(21) Appl. No.: 12/231,913

(22) Filed: Sep. 8, 2008

(51) Int. Cl. A61F 2/14

(52) U.S. Cl. 623/4.1

(57) ABSTRACT

Metal lower eyelid pocket reducer is round metal concave disk that is worn at night to reduce the size of lower eyelid pocket. The lower temperature will decrease blood circulation to the tissue and to atrophy it. Metal is a good heat conductor and radiator. When lower eyelid pocket is covered with a metal lower eyelid pocket reducer the temperature and blood supply of lower eyelid will decrease and the subcutaneous fatty tissue of lower eyelid will atrophy. This is a painless non-surgical cosmetic procedure.

### THE BACKGROUND OF THE INVENTION

When people get older the lower eyelid pocket develops. The surgical procedure is the best way to reduce it but it is costly and involves the risks of surgery. Wrinkle reduce cream claims its effectiveness but result is not guaranteed. Metal lower eyelid pocket reducer is placed onto the lower eyelid to decrease regional temperature and blood circulation of the lower eyelid to make it reduce in size.

### THE BRIEF SUMMARY OF THE INVENTION

Metal is a good conductive material for heat. The low temperature will decrease the blood circulation to the subcutaneous tissue to atrophy it. Therefore, if lower eyelid pockets are covered with a pair of metal lower eyelid reducer the temperature of lower eyelid pockets will decrease and the size of the pocket of lower eyelids will reduce.

### THE DETAILED DESCRIPTION OF THE DRAWINGS

FIG. 1 is a perspective view of metal lower eyelid pocket reducer.

FIG. 2 is the dissection view of metal lower eyelid pocket reducer.

FIG. 3 is a perspective view of a pair of metal lower eyelid pocket reducer.

### THE DETAILED DESCRIPTION OF THE INVENTION

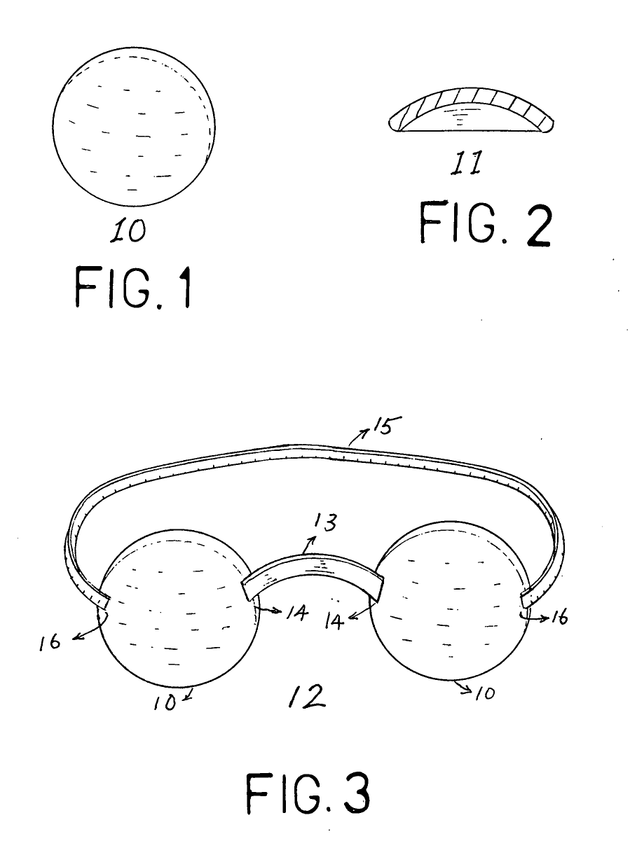

Referring now in detail to the drawings, numeral 10 of FIG. 1 is a metal lower eyelid pocket reducer. Numeral 11 of FIG. 2 is the dissection view of metal lower eyelid pocket reducer 10. Numeral 12 of FIG. 3 is a pair of metal lower eyelid pocket reducer 10. Numeral 13 of FIG. 3 is connector of a pair of metal lower eyelid pocket reducer 12. Numeral 14 of FIG. 3 is head stripe of a pair of lower eyelid pocket reducer 12. Connector 13 connects the inside 14 of each metal lower eyelid reducer 10 to make a pair of metal lower eyelid reducer 13. Numeral 15 of FIG. 3 is head stripe. Head stripe 15 connects the outside 16 of each metal lower eyelid reducer 10 for wearing on head. Metal is a good heat conductor and radiator. Metal lower eyelid pocket reducer 10 can decrease regional tissue temperature and blood circulation to lower eyelids. The poor blood circulation will cause the atrophy of subcutaneous fatty tissue of lower eyelids. To cover lower eyelids with a pair of metal lower eyelid pocket reducer 12 during sleep at night one can reduce gradually the size of lower eyelid pockets.

1. Metal lower eyelid pocket reducer comprising a round metal concave disk. The said round metal concave disk is like a contact lens and is of a variety size to fit a variety size of eyelid. The said round metal concave disk is made of any kind of metal.
